(19) United States
(12) Patent Application Publication
(10) Pub. No.: US 2026/0347770 A1
(43) Pub. Date: Nov. 5, 2026

(54) METHOD FOR PRODUCING ARTIFICIAL SKIN EQUIVALENTS COMPRISING HAIR FOLLICLES

(71) Applicant: INDUSTRY ACADEMIC COOPERATION FOUNDATION, HALLYM UNIVERSITY, Chuncheon-si (KR)

(72) Inventors: Gunyong SUNG, Seoul (KR); Subin JEONG, Seoul (KR); Hyeonmin NAM, Chuncheon-si (KR)

(73) Assignee: INDUSTRY ACADEMIC COOPERATION FOUNDATION, HALLYM UNIVERSITY, Chuncheon-si (KR)

(21) Appl. No.: 19/487,319

(22) PCT Filed: Apr. 12, 2024
(86) PCT No.: PCT/KR2024/004970

§ 371 Date: Nov. 24, 2025

(30) Foreign Application Priority Data
Apr. 12, 2024 (KR) 10-2024-0049234

(51) Int. Cl. C12N 5/071; A61L 27/24; A61L 27/26; A61L 27/36; A61L 27/38; A61L 27/56; A61L 27/60

(52) U.S. Cl. CPC C12N 5/0629; A61L 27/24; A61L 27/26; A61L 27/362; A61L 27/3808; A61L 27/3891; A61L 27/56; A61L 27/60; C12N 2502/094; C12N 2502/1323; C12N 2502/28; C12N 2510/00; C12N 2513/00; C12N 2533/54

(57) ABSTRACT

The present invention relates to a method for manufacturing an artificial skin equivalent comprising a hair follicle. The present invention provides a method for manufacturing an artificial skin equivalent, comprising: (1) preparing a spheroid; (2) injecting collagen through a lower channel pattern layer of an artificial skin equivalent manufacturing device, which comprises a substrate, a lower channel pattern layer, a scaffold, and an upper layer, to form a collagen layer; (3) injecting human umbilical vein endothelial cells (HUVECs) through the lower channel pattern layer to seed them on the collagen layer; (4) seeding by injecting fibroblasts (FBs) on the scaffold; (5) seeding the spheroid manufactured in step (1) on the scaffold; and (6) injecting keratinocytes (KCs) to seed them on the scaffold.

### TECHNICAL FIELD

The present disclosure relates to a method for manufacturing an artificial skin equivalent including hair follicles.

### BACKGROUND ART

While the number of patients receiving hair loss treatment is increasing every year, current treatments for hair loss have many limitations. Among these, treatments that relocate hair follicles (HFs) from the back of the head to areas where hair is needed do not increase the total number of hair follicles. Even when donor hair follicles are transplanted, there are often not enough hair follicles to cover the patient's hair loss area, limiting the number of patients eligible for treatment. Due to these limitations, extensive research is being conducted to apply HFs to skin-on-a-chip, which are 3D cultures of human skin equivalents under conditions similar to the human physiological environment.

Wnt is a signaling molecule that plays a central role in hair follicle morphogenesis. The presence of Wnt inhibits-catenin phosphorylation, leading to the stabilization of β-catenin, and the stabilized β-catenin is translocated to the nucleus, where β-catenin interacts with the T-cell factor (TCF)/lymphoid enhancer factor (LEF) transcription factor to act on target genes for activation. This Wnt/B-catenin signaling plays an important role in the communication between DPCs and epithelial cells. Wnt1 accelerates the progression of HF from telogen to anagen, increases hair volume, and increases the expression of hair induction-related genes such as Gli1 and LEF1. Wnt3a activates β-catenin to increase hair growth and increases the proliferation of DP cells associated with transcriptional activation. Wnt10b activates β-catenin signaling to promote the re-entry of HF from telogen to anagen and stabilizes β-catenin, thereby enhancing the differentiation of cultured primary skin epithelial cells into the hair shaft and the IRS of the HF. Wnt5a is a non-canonical Wnt expressed in the bulge and secondary hair germ cells during a telogen stage, thereby antagonizing the function of canonical Wnt. Wnt7b is important for activating and promoting the normal telogen-to-anagen transition.

However, most current models for hair follicle research fail to replicate several key features of the hair follicle microenvironment. Although animal studies are heavily reliant on hair follicle research, animal models do not fully mimic human biology. Furthermore, two-dimensional (2D) hair follicle models lose the primary inductive capacity of dermal papilla cells (DPCs) that can be observed after a certain period of time.

Transfection is a method used to introduce a gene of interest into a target cell. Electroporation, one of the non-viral transfection methods, is a method of delivering genes by briefly applying a high voltage to the cell surface, creating holes through which DNA can enter.

Therefore, the inventors have transfected DPCs to amplify Wnt signaling, which is a signaling molecule that influences hair growth, and manufactured whole skin equivalents composed of keratinocyte (KCs), fibroblasts (FBs), and human umbilical vein endothelial cells (HUVECs) to demonstrate the possibility of self-generating hair follicles on a chip.

### Prior Art Documents

(Non-Patent Document 1) BEJSOVEC, Amy. Wnt signaling: an embarrassment of receptors. Current Biology, 2000, 10.24: R919-R922.
(Non-Patent Document 2) DONG, Liang, et al. Treatment of MSCs with Wnt1a-conditioned medium activates DP cells and promotes hair follicle regrowth. Scientific Reports, 2014, 4.1:1-9.
(Non-Patent Document 3) KISHIMOTO, Jiro; BURGESON, Robert E.; MORGAN, Bruce A. Wnt signaling maintains the hair-inducing activity of the dermal papilla. Genes & development, 2000, 14.10:1181-1185.
(Non-Patent Document 4) LI, Y. -H., et al. Wnt10b promotes growth of hair follicles via a canonical Wnt signalling pathway. Clinical and experimental dermatology, 2011, 36.5: 534-540.
(Non-Patent Document 5) XING, Yi-Zhan, et al. Adenovirus-mediated Wnt5a expression inhibits the telogen-to-anagen transition of hair follicles in mice. International journal of medical sciences, 2013, 10.7:908.
(Non-Patent Document 6) KANDYBA, Eve; KOBIELAK, Krzysztof. Wnt7b is an important intrinsic regulator of hair follicle stem cell homeostasis and hair follicle cycling. Stem cells, 2014, 32.4:886-901.
(Non-Patent Document 7) INAMATSU, Mutsumi, et al. Establishment of rat dermal papilla cell lines that sustain the potency to induce hair follicles from afollicular skin. Journal of Investigative Dermatology, 1998, 111.5:767-775.

### DISCLOSURE

### Technical Problem

An object of the present invention is to provide a method for manufacturing an artificial skin equivalent including hair follicles.

Another object of the present invention is to provide an artificial skin equivalent including hair follicles.

Still another object of the present invention is to provide a method for evaluating the efficacy of a hair tonic composition using an artificial skin equivalent including hair follicles.

The objects of the present disclosure are not limited to those mentioned above, and other objects and advantages of the present disclosure not mentioned can be understood through the following description and will be more clearly understood through embodiments of the present disclosure. In addition, it will be readily apparent that the objects and advantages of the present disclosure can be realized by the means and combinations thereof set forth in the claims.

### Technical Solution

The present invention provides a method for manufacturing an artificial skin equivalent, which includes: (1) preparing a spheroid; (2) injecting collagen through a lower channel pattern layer of an artificial skin equivalent manufacturing device, which comprises a substrate, a lower channel pattern layer, a scaffold, and an upper layer, to form a collagen layer; (3) injecting human umbilical vein endothelial cells (HUVECs) through the lower channel pattern layer to seed them on the collagen layer; (4) seeding by injecting fibroblasts (FB) on the scaffold; (5) seeding the spheroid manufactured in step (1) on the scaffold; and (6) injecting keratinocytes (KC) to seed them on the scaffold.

The spheroid may include dermal papilla cells (DPCs) and keratinocytes (KCs).

The DPCs may be one or more among the DPCs transfected with LEF1, TCF1, Wnt1, or Wnt10b.

The spheroid may include dermal papilla cells (DPCs), keratinocytes (KCs) and human umbilical vein endothelial cells (HUVECs).

The DPCs may be one or more among the DPCs transfected with LEF1, TCF1, Wnt1, or Wnt10b.

The scaffold may include one or more biocompatible materials selected from the group consisting of silk, fibrin, collagen, gelatin, matrigel, alginate, chitosan, fibronectin, polyimides, hyaluronic acid, polyamic acid, polycaprolactone, polyetherimide, polystyrene, polyacrylate, polymethylmethacrylate, polylactic acid (PLA), polyglycolic acid (PGA), cellulose, nylon, polyaramid, polyvinyl alcohol, polyvinylpyrrolidone, polybenzyl glutamate, polyphenyleneterephthalamide, polyaniline, polyacrylonitrile, polyethylene oxide, a poly(lactic-co-glycolic acid) (PLGA), poly(ethylene oxide) terephthalate-co-butylene terephthalate) (PEOT/PBT), polyphosphoester (PPE), polyphosphazene (PPA), polyanhydride (PA), polytetrafluoroethylene (PTFE), polyortho ester (POE), poly(propylene fumarate)-diacrylate (PPF-DA), and poly(ethylene glycol) diacrylate (PPF-DA).

The upper layer and the lower channel pattern layer may include a polymer material.

The step (3) may be a step of inverting the artificial skin equivalent manufacturing device and injecting human umbilical vein endothelial cells (HUVECs) through the lower channel pattern layer to seed HUVECs on the collagen layer.

The artificial skin equivalent manufacturing device may include a substrate; a lower channel pattern layer formed on the substrate and having a channel through which a fluid can flow; a scaffold formed on the lower channel pattern layer and having a plurality of holes formed therein; and an upper layer formed on the scaffold, in which a medium chamber and an culture chamber are formed.

The hole formed in the scaffold may be formed by any one among a CNC machine, laser cutting, and a bio-punch.

The present invention can also provide an artificial skin equivalent manufactured by the method for manufacturing the artificial skin equivalent.

The present invention can also provide a method for evaluating the efficacy of a hair tonic composition using the artificial skin equivalent.

The method for evaluating the efficacy of a hair growth agent composition may include applying a hair growth agent composition and a control group to the artificial skin equivalent; and comparing the length of hair grown from the spheroid.

The efficacy of the hair tonic composition may be evaluated by applying the hair growth agent composition and a control group thereof to an artificial skin equivalent and comparing the rate at which hair grows from a hair follicle.

### ADVANTAGEOUS EFFECTS

The present invention can provide a method for manufacturing an artificial skin equivalent including hair follicles.

The present invention can also provide an artificial skin equivalent including hair follicles.

The present invention can also provide a method for evaluating the efficacy of a hair growth agent composition using an artificial skin equivalent including hair follicles.

### DESCRIPTION OF DRAWINGS

FIG. 1 shows a device for manufacturing an artificial skin equivalent of the present invention,

FIG. 2 shows a scaffold according to an embodiment of the present invention.

FIG. 3 shows examples of spheroids that can be manufactured using transfected DPCs and KCs.

FIG. 4 shows examples of spheroids that can be produced using transfected DPCs, KCs, and HUVECs.

FIG. 5 shows a flow chart of a method for manufacturing an artificial skin equivalent according to an embodiment of the present invention.

### MODE FOR INVENTION

Hereinafter, the present invention will be described in more detail through Examples and Experimental Examples. However, these Examples and Experimental Examples are intended to exemplify the present invention, and the scope of the present invention is not limited to these Examples and Experimental Examples.

### Device for Manufacturing Skin Equivalent Containing Hair Follicles

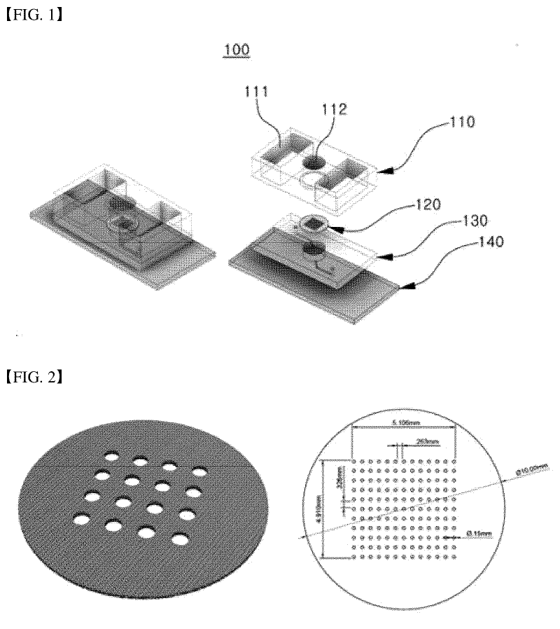

As shown in FIG. 1, the device 100 for manufacturing a skin equivalent including hair follicles according to an embodiment of the present invention consists of an upper layer 110, which consists of a medium chamber 111 made of polydimethylsiloxane (PDMS) and a culture chamber 112, a scaffold 120 having holes formed therein for positioning hair follicle spheroids so that hair can grow from the hair follicles, a lower channel pattern layer 130 made of PDMS, and a glass substrate 140.

In the PDMS, a silicone elastomer base and a silicone elastomer curing agent (Sylgard 184, K1 solution) were used at a 10:1 weight ratio.

The medium chamber is a space for storing or keeping a culture medium, and the culture chamber is a space for culturing cells. In order to manufacture an upper layer, in which two medium chambers and one culture chamber are included, an aluminum metal mold is manufactured in relief, and in order to manufacture a lower channel pattern layer, a microfluidic channel having a width of 0.2 mm and a height of 0.15 mm is engraved in relief on the aluminum metal mold. PDMS is added into the mold, and a PET film is placed on top of it so that no air bubbles are formed, and then the PDMS is flattened by pressing with a weight. After curing in an oven at 80 °C for 1 hour, the PDMS is removed from the mold, and thereby the upper layer and the lower channel pattern layer are manufacture.

As shown in FIG. 1, the surface is treated with O₂ plasma, and the glass substrate, lower channel pattern layer, scaffold, and upper layer are sequentially placed in this order, and then sterilized by treatment at 121 °C for 30 minutes in an autoclave, and dried for 24 hours to thereby manufacture the device.

A scaffold is a plate with multiple holes formed to allow hairs grown from cells can pass through. The shape is not particularly limited to circles, triangles, squares, polygons, etc., but circles are preferred. The scaffold has pores ranging from 30 μm to 100 μm, with a preferred pore size of 50 μm.

As shown in FIG. 2, the scaffold (Alvetex™) was processed into a circular pattern by laser to mark the location of the hair follicles. The holes of the scaffold can be set to have a size of 100 μm to 500 μm to be used. The holes formed in the scaffold can be formed by any one of a CNC machine, laser cutting, and a bio-punch. As the material of the scaffold, one or more biocompatible materials selected from the group consisting of silk, fibrin, collagen, gelatin, matrigel, alginate, chitosan, fibronectin, polyimides, hyaluronic acid, polyamix acid, polycarprolactone, polyetherimide, polystyrene, polyacrylate, polymethylmethacrylate, polylactic acid (PLA), polyglycolic acid (PGA), cellulose, nylon, polyaramid, polyvinyl alcohol, polyvinylpyrrolidone, polybenzyl glutamate, polyphenyleneterephthalamide, polyaniline, polyacrylonitrile, polyethylene oxide, a poly(lactic-co-glycolic acid) (PLGA), poly(ethylene oxide) terephthalate-co-butylene terephthalate) (PEOT/PBT), polyphosphoester (PPE), polyphosphazene (PPA), polyanhydride (PA), polytetrafluoroethylene (PTFE), polyortho ester (POE), poly(propylene fumarate)-diacrylate (PPF-DA), and poly(ethylene glycol) diacrylate (PPF-DA) may be used.

### Manufacture of Transfected Dermal Papilla Cells (DPCs)

(1) LEF1 transfection

Culture DPCs until they reach 70-90% confluence and harvest the cells using PBS. Add DPCs (1-2×10⁶ cells) to 100 μL of resuspension buffer R (Invitrogen), add LEF1 plasmid DNA (5-10 AL), and then transfect DPCs in accordance with an appropriate electroporation protocol using the Neon transfection system (Invitrogen). Place the transfected DPCs in a culture dish filled with Dulbecco's Modified Eagle Medium (DMEM) supplemented with Fetal Bovine Serum (FBS) and culture in an incubator at 37 °C and 5% CO₂ for two days. Cells at passages 5-8 were used.

### (2) TCF1 Transfection

Culture DPCs until they reach 70-90% confluence and harvest the cells using PBS. Add DPCs (1-2×10⁶ cells) to 100 μL of resuspension buffer R (Invitrogen), add TCF1 plasmid DNA (5-10 L), and then transfect DPCs in accordance with an appropriate electroporation protocol using the Neon transfection system (Invitrogen). Place the transfected DPCs in a culture dish filled with Dulbecco's Modified Eagle Medium (DMEM) supplemented with Fetal Bovine Serum (FBS) and culture in an incubator at 37 °C and 5% CO₂ for two days. Cells at passages 5-8 were used.

### (3) Wnt1 Transfection

Culture DPCs until they reach 70-90% confluence and harvest the cells using PBS. Add DPCs (1-2×10⁶ cells) to 100 μL of resuspension buffer R (Invitrogen), add Wnt1 plasmid DNA (5-10 μL), and then transfect DPCs in accordance with an appropriate electroporation protocol using the Neon transfection system (Invitrogen). Place the transfected DPCs in a culture dish filled with Dulbecco's Modified Eagle Medium (DMEM) supplemented with Fetal Bovine Serum (FBS) and culture in an incubator at 37 °C and 5% CO₂ for two days. Cells at passages 5-8 were used.

### (4) Wnt10b Transfection

Culture DPCs until they reach 70-90% confluence and harvest the cells using PBS. Add DPCs (1-2×10⁶ cells) to 100 μL of resuspension buffer R (Invitrogen), add Wnt10b plasmid DNA (5-10 μL), and then transfect DPCs in accordance with an appropriate electroporation protocol using the Neon transfection system (Invitrogen). Place the transfected DPCs in a culture dish filled with Dulbecco's Modified Eagle Medium (DMEM) supplemented with Fetal Bovine Serum (FBS) and culture in an incubator at 37 °C and 5% CO₂ for two days. Cells at passages 5-8 were used.

### Manufacture of Spheroids

### (1) Manufacture of DPC, KC Spheroids

Add 70% EtOH to the StemFIT 3D (Microfit Inc.) plate, remove bubbles in the well by pipetting, and fill the well with DMEM: KGM (keratinocyte growth medium) (3:1) medium and remove residual EtOH in the well by pipetting. Evenly add 5×10⁴ single cells (DPC: KC=1:1) to the center of the StemFIT 3D and culture for 15 minutes in an incubator at 37 °C and 5% CO₂. After confirming that the cells have sedimented, carefully aspirate the medium to remove any residual cells outside the well and add a new medium to prevent the remaining cells from leaking out of the well. DPCs and KCs in the StemFIT 3D are cultured in an incubator at 37 °C and 5% CO₂ to induce formation of spheroids. The culture period can be set to 3-7 days. DMEM: KGM (3:1) medium was used as the culture medium and was replaced daily.

### (2) Manufacture of Transfected DPC, KC Spheroids

Add 70% EtOH to the StemFIT 3D, remove bubbles in the well by pipetting, and fill the well with DMEM: KGM (keratinocyte growth medium) (3:1) medium and remove residual EtOH in the well by pipetting. Evenly add 5×10⁴ single cells (DPC: KC=1:1) to the center of the StemFIT 3D and culture for 15 minutes in an incubator at 37 °C and 5% CO₂. After confirming that the cells have sedimented, carefully aspirate the medium to remove any residual cells outside the well and add a new medium to prevent the remaining cells from leaking out of the well. Transfected DPCs and KCs in the StemFIT 3D are cultured in an incubator at 37 °C and 5% CO₂ to induce formation of spheroids. The culture period can be set to 3-7 days. DMEM: KGM (3:1) medium was used as the culture medium and was replaced daily. In particular, the transfected DPCs can be used as a combination of DPCs transfected with each of LEF1, TCF1, Wnt1, and Wnt10b (FIG. 3).

### (3) Manufacture of DPC, KC, HUVEC Spheroids

Add 70% EtOH to the StemFIT 3D, remove bubbles in the well by pipetting, and fill the well with DMEM: KGM(keratinocyte growth medium): EGM-2(endothelial growth medium)(3:1:4) medium and remove residual EtOH in the well by pipetting. Evenly add 5×10⁴ single cells (DPC: KC: HUVEC=1:1:1) to the center of the StemFIT 3D and culture for 15 minutes in an incubator at 37 °C and 5% CO₂. After confirming that the cells have sedimented, carefully aspirate the medium to remove any residual cells outside the well and add a new medium to prevent the remaining cells from leaking out of the well. DPCs, KCs, and HUVECs in the StemFIT 3D are cultured in an incubator at 37 °C and 5% CO₂ to induce formation of spheroids. The culture period can be set to 3-7 days. DMEM: KGM: EGM-2 (3:1:4) medium was used as the culture medium and was replaced daily.

### (4) Manufacture of Transfected DPC, KC, HUVEC Spheroids

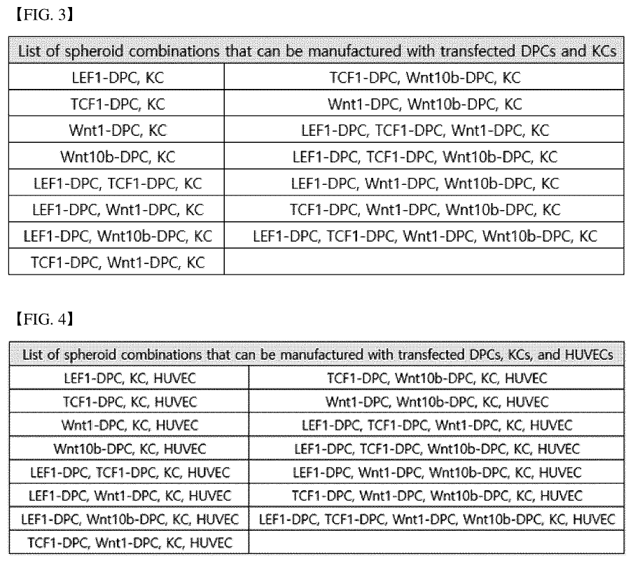

Add 70% EtOH to the StemFIT 3D, remove bubbles in the well by pipetting, and fill the well with DMEM: KGM(keratinocyte growth medium): EGM-2(endothelial growth medium)(3:1:4) medium and remove residual EtOH in the well by pipetting. Evenly add 5×10⁴ single cells (DPC: KC: HUVEC=1:1:1) to the center of the StemFIT 3D and culture for 15 minutes in an incubator at 37 °C and 5% CO 2. After confirming that the cells have sedimented, carefully aspirate the medium to remove any residual cells outside the well and add a new medium to prevent the remaining cells from leaking out of the well. Transfected DPCs, KCs, and HUVECs in the StemFIT 3D are cultured in an incubator at 37 °C and 5% CO₂ to induce formation of spheroids. The culture period can be set to 3-7 days. DMEM: KGM: EGM-2 (3:1:4) medium was used as the culture medium and was replaced daily. In particular, the transfected DPCs can be used as a combination of DPCs transfected with each of LEF1, TCF1, Wnt1, and Wnt10b (FIG. 4).

Cultivation of whole skin equivalent including hair follicle spheroids

The lower channel of the whole skin equivalent, including hair follicle spheroids, is washed with PBS and EGM-2, and the scaffold is moistened for 10 minutes in an incubator at 37 °C and 5% CO₂ after adding EGM-2 to the culture chamber.

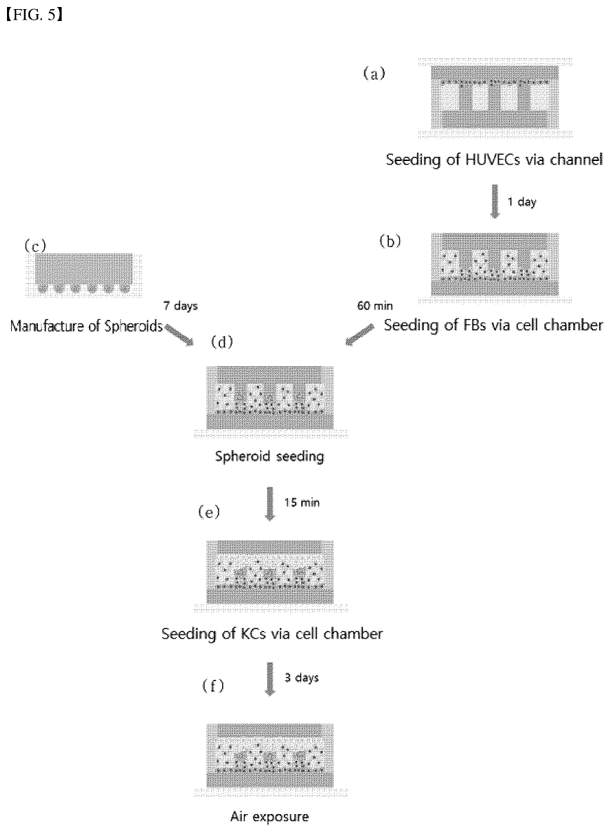

Add HUVECs (1×10⁵ cells/100 μL) through the lower channel, and fill the culture chamber and the medium chamber with EGM-2. As shown in FIG. 5(a), in a state where a sterilized glass substrate is covered on top to prevent medium leakage and inverted, culture the plate for 1 day in an incubator at 37 °C and 5% CO₂. The cells at passages 5-7 were used.

As shown in FIG. 5(b), the device is inverted again, place FBs (1×10⁶ cells/100 μL) on the scaffold, add FGM(fibroblast growth medium): EGM-2 (1:3) medium into the culture chamber, and culture for 1 hour in an incubator at 37 °C and 5% CO₂. Cells at passages 5-7 were used.

As shown in FIG. 5(d), spheroids of transfected DPCs, KCs, and HUVECs generated in the Stem FIT 3D are floated by pipetting and then allow them to enter the holes of the scaffold one by one. The spheroids are left to sediment for 15 minutes in an incubator at 37 °C and 5% CO₂, after which 3 sets of left-right and up-and-down rotations are applied at an angle of 25°and 10-second intervals to ensure good settling of the spheroids in the holes.

As shown in FIG. 5(e), after aspirating the medium, place KCs (5×10⁵ cells/100 μL) on the scaffold, and add FGM: EGM-2 (1:3) medium supplemented with 100 μg/mL of ascorbic acid to the medium chamber, and culture for 1 hour in an incubator at 37 °C and 5% CO₂. Thereafter, induce the fluid flow in the lower channel by applying an angle of 15° and 12-minute intervals for 3 days. The cells at passages 5-7 are used, and add, as the culture medium, FGM: EGM-2 (1:3) medium supplemented with 100 ug/mL of ascorbic acid into the medium chamber, and add KGM supplemented with 1.5 mM calcium chloride into the culture chamber and replace daily.

As shown in FIG. 5(f), E-media: EGM-2 (3:1) medium supplemented with 100 μg/mL of L-ascorbic acid and 1.5 mM calcium chloride is then added only to the medium chamber in the air-liquid interface state, and culture for 4 weeks at an angle of 15°at 12-minute intervals.

### DETAILED DESCRIPTION OF MAIN ELEMENTS

100: Device for manufacturing skin equivalent including hair follicles; 110: upper layer; 120: scaffold; 130: lower channel pattern layer; 140: substrate; 111: medium chamber; 112: culture chamber

1. A method for manufacturing an artificial skin equivalent, comprising:
(1) preparing a spheroid;
(2) injecting collagen through a lower channel pattern layer of an artificial skin equivalent manufacturing device, which comprises a substrate, a lower channel pattern layer, a scaffold, and an upper layer, to form a collagen layer;
(3) injecting human umbilical vein endothelial cells (HUVECs) through the lower channel pattern layer to seed them on the collagen layer;
(4) seeding by injecting fibroblasts (FB) on the scaffold;
(5) seeding the spheroid manufactured in step (1) on the scaffold; and
(6) injecting keratinocytes (KC) to seed them on the scaffold.

2. The method of claim 1, wherein the spheroid comprises dermal papilla cells (DPCs) and keratinocytes (KCs).

3. The method of claim 2, wherein the DPCs are one or more among the DPCs transfected with LEF1, TCF1, Wnt1, or Wnt10b.

4. The method of claim 1, wherein the spheroid comprises dermal papilla cells (DPCs), keratinocytes (KCs), and human umbilical vein endothelial cells (HUVECs).

5. The method of claim 4, wherein DPCs are one or more among the DPCs transfected with LEF1, TCF1, Wnt1, or Wnt10b.

6. The method of claim 1, wherein the scaffold comprises one or more biocompatible materials selected from the group consisting of silk, fibrin, collagen, gelatin, matrigel, alginate, chitosan, fibronectin, polyimides, hyaluronic acid, polyamic acid, polycaprolactone, polyetherimide, polystyrene, polyacrylate, polymethylmethacrylate, polylactic acid (PLA), polyglycolic acid (PGA), cellulose, nylon, polyaramid, polyvinyl alcohol, polyvinylpyrrolidone, polybenzyl glutamate, polyphenyleneterephthalamide, polyaniline, polyacrylonitrile, polyethylene oxide, a poly(lactic-co-glycolic acid) (PLGA), poly(ethylene oxide) terephthalate-co-butylene terephthalate) (PEOT/PBT), polyphosphoester (PPE), polyphosphazene (PPA), polyanhydride (PA), polytetrafluoroethylene (PTFE), polyortho ester (POE), poly(propylene fumarate)-diacrylate (PPF-DA), and poly(ethylene glycol) diacrylate (PPF-DA).

7. The method of claim 1, wherein the upper layer and the lower channel pattern layer comprise a polymer material.

8. The method of claim 1, wherein step (3) is a step of inverting the artificial skin equivalent manufacturing device and injecting human umbilical vein endothelial cells (HUVECs) through the lower channel pattern layer to seed HUVECs on the collagen layer.

9. The method of claim 1, wherein the artificial skin equivalent manufacturing device comprises:
a substrate;
a lower channel pattern layer formed on the substrate and having a channel through which a fluid can flow;
a scaffold formed on the lower channel pattern layer and having a plurality of holes formed therein; and
an upper layer formed on the scaffold, in which a medium chamber and a culture chamber are formed.

10. The method of claim 1, wherein the hole formed in the scaffold is formed by any one among a CNC machine, laser cutting, and a bio-punch.

11. An artificial skin equivalent manufactured by the method for manufacturing of an artificial skin equivalent of claim 1.

12. A method for evaluating the efficacy of a hair growth agent composition using the artificial skin equivalent of claim 11.

13. The method of claim 12, comprising:
applying a hair growth agent composition and a control group to the artificial skin equivalent; and
comparing the length of hair grown from the spheroid.
